(19) United States
(12) Patent Application Publication
(10) Pub. No.: US 2010/0034736 A1
(43) Pub. Date: Feb. 11, 2010

(54) IDENTIFICATION OF CANCER STEM CELLS USING GENETIC MARKERS

(75) Inventors: Isidro Sanchez-Garcia, Salamanca (ES); Maria Perez-Caro, Salamanca (ES)

Correspondence Address: MORRISON & FOERSTER LLP, 425 MARKET STREET, SAN FRANCISCO, CA, 94105-2482, US

(73) Assignee: Consejo Superior de Investigaciones Cientificas, Salamanca (ES)

(21) Appl. No.: 12/440,369

(22) PCT Filed: Sep. 7, 2007
(86) PCT No.: PCT/IB2007/003648

§ 371 Date: Sep. 15, 2009

(30) Foreign Application Priority Data
Sep. 7, 2006 (EP) 06018771.3

(51) Int. Cl. A61K 51/10; C12Q 1/68; C12N 5/00; C12Q 1/02; G01N 33/53; C07H 21/00; A01K 67/027; A61K 39/395; A61K 31/7052; A61K 38/02; A61P 35/00

(52) U.S. Cl. 424/1.49; 435/6; 435/375; 435/29; 435/7.1; 536/24.1; 800/13; 800/9; 800/10; 435/325; 424/174.1; 514/44 R; 514/2

(57) ABSTRACT

The invention relates to the identification of markers for cancer stem cells. These markers can be used in a number of different ways, including diagnosis and therapy. In particular, the invention relates to a method of detecting, identifying and/or quantifying cancer stem cells, the method comprising the step of assessing the level of expression; the activity; or the sequence of the SLUG, OVOL1 and/or OVOL2 gene, promoter and/or expression product in a cell.

The invention relates to the identification of markers for cancer stem cells. These markers can be used in a number of different ways, including diagnosis and therapy.

All publications, patents and patent applications cited herein are incorporated in full by reference.

### BACKGROUND

The conventional view of cancer is that this disease is caused by fast-growing highly mutant cells caused by multi-step mutation events at a cellular level. Current treatments are directed towards the eradication of this population of cells, either by chemotherapy, irradiation. In addition, more complex immunotherapies and gene therapies are emerging. All these methods eradicate a significant volume of the tumour mass by targeting the neoplastic, highly proliferative cells making up its volume. When these methods fail, it is perceived that this is because some cells survived the therapy process, either because of inadequacies in the treatment or because a proportion of cells become resistant.

The concept that cancers arise from stem cells has recently been given new impetus by advances in stem cell biology. This hypothesis holds that tumours originate in stem cells through dysregulation of the tightly regulated process of self-renewal. Tumours thus retain a subcomponent of cells that retain key stem cell properties, including the ability to self-renew. This drives tumourigenesis and aberrant differentiation. This theory has gained ground among many researchers in this area (Wicha et al 2006 and references cited therein; see also U.S. Pat. No. 6,004,528) although others remain unconvinced (Hill et al., 2006).

The implications of this hypothesis are wide-ranging and fundamental, having ramifications for cancer risk assessment, early detection, prognostication and development. It also casts doubt on and perhaps explains the failure of current anti-cancer therapeutics which target the end stage differentiated cancer cells rather than the cancer stem cells from which they are derived.

The cancer stem cell hypothesis requires that a rare population of cancer stem cells be targeted. Of course, this carries with it a problem, in that the body's healthy stem cell population must be spared. In particular, the stem cell model has important implications for the development of markers for the early detection of cancer, since this model holds that important prognostic and predictive information can be obtained from identifying cancer stem cells when they arise. If markers can be developed that distinguish between cancer stem cells and their healthy counterparts, such markers may also be used to target the causative diseased cells and thus reduce cancer stem cell numbers. In particular, therapeutic interventions that either induce apoptosis or differentiation with a loss of self-renewal capacity in these cells represents a rational therapeutic approach to cancer prevention.

There is thus a great need for a method that specifically and selectively identifies cancer stem cells and which differentiates these from healthy stem cells.

### SUMMARY OF THE INVENTION

Without wishing to be bound by theory, the inventors hypothesised that cancer stem cells share characteristics with embryonic stem cells. These characteristics include, among others, the capacity for self renewal and the expression of specific cell surface markers. The inventors have therefore now identified SLUG, OVOL1 and OVOL2 as biomarkers for cancer stem cells. Expression of these genes has been identified in cancer stem cells in the peripheral blood of humans with cancer and in the peripheral blood of mouse models of human cancer, but not in the peripheral blood of healthy humans or mice.

Accordingly, expression of these biomarkers may be exploited to identify the existence of cancer stem cells in a patient. This is advantageous over current methods of cancer diagnosis such as blood tests, and radiologic studies, which require relatively high numbers of cells. Accordingly, these diagnostic tests allow detection of a tumour or potential cancer at a much earlier stage than is possible now. According to this aspect of the invention, there is provided a method to detect, identify and/or quantify cancer stem cells, the method comprising the step of assessing: (a) the level of expression; (b) the activity; or (c) the sequence of the SLUG, OVOL1 and/or OVOL2 gene, promoter and/or expression product in a cell. Preferably, the cells are haematopoietic, epidermal, breast, ovary, lung, pancreas, prostate, brain, colon, bone marrow, and/or lymph cancer stem cells.

The invention also provides a method for diagnosing cancer and/or assessing the stage and/or severity of the cancer in a patient comprising the step of assessing: (a) the level of expression; (b) the activity; or (c) the sequence of the SLUG, OVOL1 and/or OVOL2 gene, promoter and/or expression product in a biological sample from the patient and comparing the level of expression, activity or sequence to a control level, activity or sequence, wherein a difference compared to said control is indicative of cancer disease or a predisposition to cancer.

The SLUG gene, also known as SNAI2, is represented in GenBank as the accession number NM_{—}003068 (mRNA) and NP_{—}003059 (protein) [see http://www.ncbi.nlm.nih.gov/entrez/query.fcgi?db=gene&cmd=Retrieve&dopt=full_report&list_uids=6591]. The OVOL1 gene is represented in GenBank as the accession number NM_{—}004561 (mRNA) and NP_{—}004552 (protein) [see http://www.ncbi.nlm.nih.gov/entrez/query.fcgi?db=gene&cmd=Retrieve&dopt=full_report&list_uids=5017]. The OVOL2 gene is represented in GenBank as the accession number NM_{—}021220 (mRNA) and NP_{—}067043 (protein) [see http://www.ncbi.nlm.nih.gov/entrez/query.fcgi?db=gene&cmd=Retrieve&dopt=full_report&list_uids=58495].

Expression of these biomarkers will also act as a target for therapies directed at selectively destroying the cancer stem cell compartment. Such therapies may target markers present on the cancer stem cells, for example, for the purpose of destroying these cells. This might be done by inducing terminal differentiation, apoptosis or programmed cell death. This may perhaps be effected by inducing a switch from a symmetric proliferative mitotic program to asymmetric cell division or a terminal differentiation/apoptotic program. By targeting the immortal population of cells within a tumour, these therapies are likely to be more successful than conventional therapies in reducing the number of cancer cells, and the patient is also less likely to suffer a relapse.

This aspect of the invention thus provides a method for the eradication of cancer stem cells, the method including the step of (a) immunotherapy directed at cancer stem cells by targeting SLUG, OVOL1 and/or OVOL2 expression products or their coding genes or promoters; or (b) inducing a switch in cancer stem cells either to effect a change from exponential to non-exponential cell growth or to induce a differentiation or apoptotic program by targeting SLUG, OVOL1 and/or OVOL2 expression products or their coding genes or promoters. Such methods may target factors that are implicated in these changes, but must also be specific to cancer stem cells.

Expression of these biomarkers may also used as a tool to model human cancer and/or other stem cell derived diseases in mice. For example, in a model system, a promoter of one of these biomarkers, for example, a promoter of SLUG, OVOL1 and/or OVOL2, can be operatively linked to a heterologous disease-related gene and thus be used to control the expression of such heterologous disease-related genes. Examples of disease-related genes include oncogenes, such as the genes identified in the art as BCR-ABLp210, BCR-ABLp190, Slug, Snail, HOX11, RHOM2/LMO-2, TAL1, Maf-B, FGFR, c-maf, MMSET, BCL6, BCL10, MALT1, cyclin D1, cyclin D3, SCL, LMO1, LMO2, TEL-AML1, E2A-HLF, E2A-Pbx1, TEL-ABL, AML1-ETO, FUS-CHOP, FUS-DDIT3, EWS-WT1, EWS FLI1, EWSR1-DDIT3, FUS-ATF1, FUS-BBF2H7, K-RASv12, Notch1, etc. A comprehensive lists of oncogenes are provided at: http://www.infobiogen.fr/services/chromcancer/Genes/Geneliste.html; see also Cooper G. Oncogenes. Jones and Bartlett Publishers, 1995; Vogelstein B, Kinzler K W. The Genetic Basis of Human Cancer. McGraw-Hill: 1998; http://cancerquest.org/index.cfm?page=780. This aspect of the invention therefore provides an isolated nucleic acid construct that comprises the promoter of SLUG, OVOL1 and/or OVOL2 operatively linked to an oncogene.

A promoter of one of the biomarkers described herein can also be used to control the expression of a reporter gene. Reporter genes are nucleic acid sequences encoding directly or indirectly assayable proteins. They are used to replace other coding regions whose protein products are unsuitable or not amenable to the assay envisaged. Examples of suitable reporter genes that are known in the art and may be used in the present invention are selected from those genes encoding proteins including but not limited to: chloramphenicol-acetyltransferase, β-galactosidase, β-glucuronidase, luciferase, β-galactosidase, fluorescent proteins (GFP, YFP, RFP, etc.), secreted alkaline phosphatase (SEAP), major urinary protein (MUP) or human chorionic gonadotrophin (hCG). It will be understood that the above list of suitable reporter genes is not exhaustive or exclusive and is not intended to limit the scope of the application. The person skilled in the art may select another reporter system which will equally be applicable to the present invention. The invention therefore provides an isolated nucleic acid construct that comprises the promoter of SLUG, OVOL1 and/or OVOL2 operatively linked to a reporter gene.

One aspect of this invention also provides a transgenic non-human animal, hereinafter referred to as the transgenic non-human animal of the invention. The non-human animal that is termed “transgenic” comprises a transgene in its genome. According to the invention, said transgene comprises a heterologous nucleic acid construct that comprises the SLUG, OVOL1 and/or OVOL2 promoter and/or coding sequence. The transgene may also comprise a heterologous gene such as an oncogene or a reporter gene that is operatively linked to a promoter of SLUG, OVOL1 and/or OVOL2.

Transgenic non-human animals that express a transgene that comprises the promoter of SLUG, OVOL1 and/or OVOL2 operatively linked to a reporter gene can be used to image the expression of the biomarker in the transgenic non-human animal model. For example, both the temporal aspects of biomarker expression and the tissue distribution of biomarker expression can be monitored in this manner. This aspect of the invention thus provides a method of detecting cancer stem cells in a transgenic non-human animal, comprising the step of identifying the cells in the above-described transgenic animal that express the reporter gene.

The invention also allows cancer stem cells to be isolated, enriched and purified from a patient or population of patients. This allows the development of assays to identify factors influencing cancerous growth in cancer stem cells, to analyse populations of cancer stem cells for patterns of gene expression or protein expression, to identify new anticancer drug targets, to predict the sensitivity of cancer stem cells to existing and/or new therapies, and generally to model cancer development and treatment. According to this aspect of the invention, there is provided a method of identifying cancer stem cells, the method comprising assessing the level of expression, activity or sequence of the SLUG, OVOL1 and/or OVOL2 gene and/or expression product in a cell.

Such methods may exploit the properties of monoclonal antibodies. For example, mAbs specific for cancer stem cells may be modified so as to be attached directly or indirectly to a therapeutic moiety such as an enzyme, chemotherapeutic drug, cytotoxin, radionuclide or cytokine.

The invention also relates to purified cancer stem cells. Accordingly, one aspect of the invention relates to a substantially pure culture of cancer stem cells wherein said cells express SLUG, OVOL1 and/or OVOL2 at a significant level. The cells may be any kind of cancer stem cells, such as, for example cells derived from an epithelial cancer and/or mesenchymal cancer. The term “epithelial cancer”, as used herein, refers to a cancer of which tumour cells are the cells that line the internal and external surfaces of the body. The term “mesenchymal cancer”, as used herein, refers to a cancer which tumour cells develop into connective tissue, blood vessels and lymphatic tissue. Illustrative, non-limitative examples of said epithelial or mesenchymal cancers include lymphomas, leukaemias, sarcomas and carcinomas, such as, for example, chronic myeloid leukaemia, B-cell acute lymphoblastic leukaemia, T-cell acute lymphoblastic leukaemia, acute myeloid leukaemia, chronic myeloid leukaemia, lymphoproliferative syndromes, multiple myeloma, liposarcoma, and Ewing sarcoma (Best and Taylor. Bases fisiológicas de la patología médica. Madrid: Editorial Médica Panamericana, 12th ed., 1993). As the skilled person will understand, this is not an exhaustive list.

The cancer stem cells may be any vertebrate cancer stem cells, but of greatest interest are animal stem cells, including mammalian cells, both human and non-human primate cells, and rodent cells, in particular mouse, rat or hamster cells. Preferably, at least 50% of the cells in the substantially pure culture are cancer stem cells that express at least one of SLUG, OVOL1 and OVOL2, and this proportion may be more, such as 60%, 70%, 80%, 90%, 95% or more. The substantially pure culture of cancer stem cells will preferably contain less than 50% of cancer cells that are not cancer stem cells, and this proportion may be less, for example, 25%, 10%, 5%, 1% or less.

In an alternative manner of expressing the substantially pure nature of the culture of cancer stem cells according to the invention, cancer stem cells may be 5-fold enriched, 25-fold enriched, 50-fold enriched or more for cancer stem cells that express at least one of SLU, OVOL1 and OVOL2, relative to other types of cell as compared to an untreated biological sample obtained directly from a patient or from a culture of cells. Such other cell types include non-stem cancer cells, non-cancerous stem cells and other, healthy cells that are present in the body or in in vitro culture.

The cancer stem cells according to the invention are distinguished by markers, such as the SLUG, OVOL1 and/or OVOL2 biomarkers identified herein.

In addition, such cancer stem cells may express molecules that are specific for stem cell lineages, such as Sca1 in the mouse. Other useful biomarkers include CD34, CD44 and CD38, human epithelial antigen (HEA) in humans, CD133, carcinoembryonic antigen (CEA) in humans, α2β1 integrin, and Lrig1 in humans and mice. The cancer stem cells of the invention may have multilineage (lymphoid and myeloid) developmental potential. The cancer stem cells of the invention preferably express significant levels of telomerase.

Additionally, the cancer stem cells of the invention preferably do not express significant levels of one or more mature lineage markers selected from the group consisting of CD2, CD3, CD4, CD7, CD8, CD10, CD11b, CD14, CD15, CD16, CD19, CD20, CD31, CD45, CD56, CD64, CD140b and glycophorin A (GPA) in humans. The cancer stem cells of the invention preferably do not express CD24 or express low levels of CD24. In the case of murine cells, the cancer stem cells of the invention are Lin− and do not express mature lineage markers.

By “significant levels” as used herein is mean a level of expression and/or activity that is 5%, 10%, 20%, 30%, 40%, 50%, 100%, 200% or more greater than the level of expression and/or activity in a control cell.

The cancer stem cells of the invention may be isolated from a patient. The patient may be an individual diagnosed with cancer, an individual considered at risk from suffering cancer, an individual suspected of having cancer, an individual not suspected of having cancer and who gives an outward impression of being in good health, or an individual taking part in drug and/or diagnostic development clinical trials.

In an alternative, the cancer stem cells of the invention may be grown in culture. The cells may be isolated from an animal model, such as a mouse model of the type described in international patent application PCT/IB2006/001969. In addition to expression of SLUG, OVOL1 and/or OVOL2, and Sca1 in the mouse, cancer stem cells isolated from a cancer model animal may contain a chromosomal anomaly that is associated with cancer. Such a chromosomal anomaly may include an oncogene, as previously defined above.

The invention also embraces methods of isolating cancer stem cells. Such methods involve the selective enrichment of cancer stem cells which express the SLUG, OVOL1 and/or OVOL2 gene. Suitable methods for the enrichment of cells expressing a marker such as SLUG, OVOL1 and/or OVOL2 are known in the prior art and include centrifugation based methods, elutriation, density gradient separation, apheresis, affinity selection, panning, immunological-based systems such as fluorescence activated cell sorting (FACS); immunoaffinity exchange; non-optical cell sorting methods including magnetic cell sorting using antibody-coated magnetic particles that bind to a specific cell type to separate desired cells.

The invention also relates to methods for propagating cancer stem cells of the invention. Such a method may involve exposing cancer stem cells in culture to a concentration of lysate produced from cells of at least one selected differentiated cell type, the concentration able to induce the cancer stem cells to propagate by preferentially undergoing either symmetric mitosis, whereby each dividing cancer stem cell produces two identical daughter cancer stem cells, or asymmetric mitosis, whereby each dividing cancer stem cell produces one identical daughter cancer stem cell and one daughter cell that is more differentiated than the cancer stem cells.

The invention also relates to methods for diagnosing cancer, assessing the stage and/or severity of the disease. Such a method may comprise screening a subject for cancer, or a predisposition to cancer, comprising the steps of testing biological a sample from the subject for the presence of cancer stem cells. A method of this type may include the steps of detecting cells expressing a product of the SLUG, OVOL1 and/or OVOL2 gene. A level of expression on cells that is different to a control level is indicative of the presence of cancer stem cells and is also indicative of cancer. A method of this type may include the steps of detecting the activity of an expression product of the SLUG, OVOL1 and/or OVOL2 gene.

The methods of the invention are particularly useful in detecting the early stages of cancer in outwardly healthy individuals. However, the methods of the invention can be used to diagnose cancer, and/or to assess the stage and/or severity of the disease in any patient. The patient may be an individual diagnosed with cancer, an individual considered at risk from suffering cancer, an individual suspected of having cancer, an individual not suspected of having cancer and who gives an outward impression of being in good health, or an individual taking part in drug and/or diagnostic development clinical trials. When the individual is taking part in a clinical trial, the method can be used to monitor the clinical trial. The patient may or may not be receiving treatment for cancer or any other disease. The method of the invention can also be used to screen for the recurrence of cancer in an individual that has previously been diagnosed with cancer but at the time of the screening is outwardly healthy.

The invention also provides methods for assessing the progression of cancer in a patient comprising comparing the expression products of the SLUG, OVOL1 and/or OVOL2 gene referred to above in a biological sample at a first time point to the expression of the same expression product at a second time point, wherein the increase or decrease in expression, or in the rate of increase or decrease of expression, at the second time point relative to the first time point is indicative of the progression or remission of the cancer.

In particular, the invention relates to a method for assessing the progression of cancer in a patient, the method comprising comparing the expression products of the SLUG, OVOL1 and/or OVOL2 gene referred to above in a biological sample at a first time point to the expression of the same expression product at a second time point, wherein the patient receives therapy between the first and second time points. In this aspect, the invention provides a method for assessing the response of a patient to therapy. The invention also provides a method for prognostication of cancer, the method comprising comparing the expression products of the SLUG, OVOL1 and/or OVOL2 gene referred to above in a biological sample from said patient at a first time point to the expression of the same expression product at a second time point, wherein the increase or decrease in expression, or in the rate of increase or decrease of expression, at the second time point relative to the first time point can be an indication of the prognosis.

In any of the methods described above, the increase or decrease in the level of the expression product may be 2-fold, 3-fold, 5-fold, 10-fold, 20-fold, 50-fold, or even 100-fold or more.

The expression product is preferably a protein, although alternatively mRNA expression products may also be detected.

If a protein is used, the protein is preferably detected by an antibody which preferably binds specifically to that protein. The term “binds specifically” means that the antibodies have substantially greater affinity for their target polypeptide than their affinity for other related polypeptides. Preferably, the anti-SLUG antibody is specific for SLUG and does not cross react with other members of the SNAIL family or related splice variants of SLUG. Similarly, the anti-OVOL1 antibody should be specific for OVOL1 and the anti-OVOL2 antibody should be specific for OVOL2 and these should not cross react with other members of the OVOL family or related splice variants of OVOL1 and/or OVOL2. Alternatively, the anti-SLUG, anti-OVOL1 and/or anti-OVOL2 antibody may bind to all splice variants, deletion, addition and/or substitution mutants of SLUG, OVOL1 or OVOL2 respectively.

The anti-SLUG, anti-OVOL1 and anti-OVOL2 antibodies may, respectively, be specific for the SLUG, OVOL1 and OVOL2 extracellular domains. The antibodies may be specific for cancer associated SLUG, OVOL1 and/or OVOL2 proteins as these are expressed on or within cancerous cells. For example, glycosylation patterns in cancer-associated proteins as expressed on cancer stem cells may be different to the patterns of glycosylation in these same proteins as these are expressed on non-cancerous cells. Preferably, in such a scenario, antibodies according to the invention are specific for cancer-associated proteins as expressed on cancerous cells only. This is of particular value for therapeutic antibodies.

As used herein, the term “antibody” refers to intact molecules as well as to fragments thereof, such as Fab, F(ab′)2 and Fv, which are capable of binding to the antigenic determinant in question. By “substantially greater affinity” we mean that there is a measurable increase in the affinity for the target polypeptide of the invention as compared with the affinity for other related polypeptides. Preferably, the affinity is at least 1.5-fold, 2-fold, 5-fold 10-fold, 100-fold, 10³-fold, 10⁴-fold, 10⁵-fold, 10⁶-fold or greater for the target polypeptide.

Preferably, the antibodies bind to SLUG, OVOL1 and/or OVOL2 with high affinity, preferably with a dissociation constant of 10⁻⁴M or less, preferably 10⁻⁷M or less, most preferably 10⁻⁹M or less; subnanomolar affinity (0.9, 0.8, 0.7, 0.6, 0.5, 0.4, 0.3, 0.2, 0.1 nM or even less) is preferred.

Monoclonal antibodies to SLUG, OVOL1 and/or OVOL2 polypeptides can be readily produced by one skilled in the art. The general methodology for making monoclonal antibodies using hybridoma technology is well known (see, for example, Kohler, G. and Milstein, C., Nature 256: 495-497 (1975); Kozbor et al., Immunology Today 4: 72 (1983); Cole et al., 77-96 in Monoclonal Antibodies and Cancer Therapy, Alan R. Liss, Inc. (1985). Other relevant texts include Goding, Monoclonal Antibodies: Principles and Practice, Academic Press, (1986) pp. 59-103; Waldmann, T. A. (1991) Science 252:1657-1662.

Chimeric antibodies, in which non-human variable regions are joined or fused to human constant regions (see, for example, Liu et al, Proc. Natl. Acad. Sci. USA, 84, 3439 (1987)), may also be of use.

The antibody may be modified to make it less immunogenic in an individual, for example by humanisation (see Jones et al., Nature, 321, 522 (1986); Verhoeyen et al., Science, 239, 1534 (1988); Kabat et al., J. Immunol., 147, 1709 (1991); Queen et al., Proc. Natl. Acad. Sci. USA, 86, 10029 (1989); Gorman et al., Proc. Natl Acad. Sci. USA, 88, 34181 (1991); and Hodgson et al., Bio/Technology, 9, 421 (1991)). The term “humanised antibody”, as used herein, refers to antibody molecules in which the CDR amino acids and selected other amino acids in the variable domains of the heavy and/or light chains of a non-human donor antibody have been substituted in place of the equivalent amino acids in a human antibody. The humanised antibody thus closely resembles a human antibody but has the binding ability of the donor antibody.

The antibody may be modified by the addition of a detectable label, such as a radiolabel, a fluorescent label, biotin, strepatvidin or an enzyme label such as horseradish peroxidase HRP. Radiolabeld monoclonal antibodies can be used to make radiotracers for use in molecular imaging of cancer stem cells.

In a further alternative, the antibody may be a “bispecific” antibody, that is, an antibody having two different antigen binding domains, each domain being directed against a different epitope. In the present case, one of the binding specificities may be for the SLUG, OVOL1 and/or OVOL2 polypeptide, or a fragment thereof, the other one is for any other antigen, and preferably for a cell-surface protein or receptor or receptor subunit that is also expressed on cancer stem cells.

Where SLUG, OVOL1 and/or OVOL2 mRNA expression product is used, it is preferably detected by the steps of contacting a tissue sample with a probe under stringent conditions that allow the formation of a hybrid complex between the mRNA and the probe; and detecting the formation of a complex. Other methods known in the art may also be used to detect the SLUG, OVOL1 and/or OVOL2 mRNA expression product including, but not limited to, Northern blotting, RT-PCR, and quantitative RT-PCR.

Cancer associated genes themselves may be detected by contacting a biological sample with a nucleic acid probe under stringent conditions that allow the formation of a hybrid complex between a nucleic acid expression product encoding the SLUG, OVOL1 and/or OVOL2 gene and the probe; and detecting the formation of a complex between the probe and the nucleic acid from the biological sample.

Preferred methods include comparing the amount of complex formed with that formed when a control tissue is used e.g. healthy stem cells, wherein a difference in the amount of complex formed between the control and the sample indicates the presence of cancer or a predisposition to cancer. Preferably the difference between the amount of complex formed by the test tissue compared to the normal tissue is an increase. More preferably a two-fold increase in the amount of complex formed is indicative of disease. Even more preferably, a 3-fold, 4-fold, 5-fold, 10-fold, 20-fold, 50-fold or even 100-fold increase in the amount of complex formed is indicative of disease.

The biological sample used in any of the methods of the invention is preferably a tissue sample. Any tissue sample may be used. The tissue samples for use in the methods of the invention may be obtained from a variety of sources, preferably blood, although in some instances samples such as breast, ovary, lung, pancreas, prostate, brain, colon, bone marrow, lymph, cerebrospinal fluid, synovial fluid, and the like may be used. Such samples can be separated by centrifugation, elutriation, density gradient separation, apheresis, affinity selection, panning, FACS, centrifugation with Hypaque, etc. prior to analysis, and usually a mononuclear fraction (PBMC) will be used. Once a sample is obtained, it can be used directly, frozen, or maintained in appropriate culture medium for short periods of time. Various media can be employed to maintain cells. The samples may be obtained by any convenient procedure, such as the drawing of blood, venipuncture, biopsy, or the like. Usually a sample will comprise at least about 10² cells, more usually at least about 10³ cells, and preferable 10⁴, 10⁵ or more cells. Typically the samples will be from human patients, although animal models, including the non-human transgenic animal models of the invention, may find use, e. g. equine, bovine, porcine, canine, feline, primate or rodent, e. g. mice, rats, and hamster models.

The methods of the invention may also be used to localise the cancer. Detection of cancer stem cells in a tissue sample is indicative of cancer in that tissue. For example, detection of an expression product of the SLUG, OVOL1 and/or OVOL2 gene in a breast tissue sample might be indicative of cancer in that tissue.

The invention also encompasses methods to image cancer including imaging methods based on, for example, magnetic resonance (MR), x-ray computed tomography (CT), single photon emission computed tomography (SPECT) and optical coherence tomography (OCT), positron emission tomography (PET), and quantitative autoradiography (QAR).

Radiolabels, including radiolabelled monoclonal antibodies that are specific SLUG, OVOL1 and/or OVOL2 and radiolabelled nucleic acid probes that hybridise specifically with the SLUG, OVOL1 and/or OVOL2 mRNA, coding sequence and/or promoter sequence, can be used as radiotracers in combination with known imaging methods, such as the methods described above, to detect and localise cancer stem cells. Using microimaging techniques such as micoPET and microCT in combination with the radiotracers that target SLUG, OVOL1 and/or OVOL2, it is envisaged that imaging of tumours down to the single cell level could be achieved.

Any radiolabel can be used in the imaging methods of the invention. In particular isotopes with short half lives such as ^{99m}Tc, ¹¹C, ¹³N, ¹⁵O, and ¹⁸F are suitable for use in the imaging methods described above.

Thus, the invention provides methods to image and localise cancer stem cells in a patient. The imaging methods of the invention can be used to diagnose cancer, and/or to assess the stage and/or severity of the disease in any patient, and to monitor the progression or remission of cancer. The imaging methods can also be used to monitor the effect of therapy on the stage and/or severity of cancer and to monitor clinical trials.

Once cancer stem cells have been localised using the imaging methods described above, they can be isolated and purified as described above,

The invention also provides kits useful for diagnosing cancer comprising an antibody that binds to an expression product of the SLUG, OVOL1 and/or OVOL2 gene; and a reagent useful for the detection of a binding reaction between said antibody and said polypeptide. Preferably, the antibody binds specifically to the SLUG, OVOL1 and/or OVOL2 polypeptide.

Furthermore, the invention provides kits useful for diagnosing cancer comprising a nucleic acid probe that hybridises under stringent conditions to the SLUG, OVOL1 and/or OVOL2 gene; primers useful for amplifying the SLUG, OVOL1 and/or OVOL2 gene; and optionally instructions for using the probe and primers for facilitating the diagnosis of disease.

The invention further provides antibodies, nucleic acids, or proteins suitable for use in modulating the expression of an expression product of the SLUG, OVOL1 and/or OVOL2 gene, for use in isolating cancer stem cells, and thus, for treating cancer.

The invention further provides assays for identifying a candidate agents that modulate the growth and/ or development of a cancer stem cell, comprising:

a) detecting the level of expression of an expression product of the SLUG, OVOL1 and/or OVOL2 gene or promoter in the presence of the candidate agent; and

b) comparing that level of expression with the level of expression in the absence of the candidate agent, wherein a reduction in expression indicates that the candidate agent modulates the level of expression of the expression product of the SLUG, OVOL1 and/or OVOL2 gene or promoter.

The invention also provides methods for identifying agents that modify the expression level of the SLUG, OVOL1 and/or OVOL2 gene, comprising:

a) contacting a cell expressing the SLUG, OVOL1 and/or OVOL2 gene or promoter as defined in any of the above-described embodiments of the invention with a candidate agent, and

b) determining the effect of the candidate agent on the cell, wherein a change in expression level indicates that the candidate agent is able to modulate expression.

Preferably, a cell used for this assay belongs to a cell type in which the SLUG, OVOL1 and/or OVOL2 protein is implicated as having a role in causing cancer. The transgenic models of the invention, described above, in which a reporter gene is operatively linked to one or more of SLUG, OVOL1 and OVOL2, are also of particular utility in identifying agents that are effective in the above assay methods.

Preferably the agent is a polynucleotide, a polypeptide, an antibody or a small organic molecule.

The invention also provides the use of agents identified by the above methods for treating cancer.

Accordingly, the invention provides methods for treating cancer in a patient, comprising reducing the number of cancer stem cells that express a product of the SLUG, OVOL1 and/or OVOL2 gene. Such a method preferably comprises administering to the patient an antibody, a nucleic acid, a polypeptide or agent identified in the above methods in a therapeutically-effective amount sufficient to target cancer stem cells for destruction.

The invention therefore also provides the use of an antibody, a nucleic acid, a polypeptide or agent identified in the above methods that binds to or modulates the level of an expression product of the SLUG, OVOL1 and/or OVOL2 gene, in the manufacture of a medicament for the treatment or diagnosis of cancer. Such level of expression is preferably modulated by action on the gene, mRNA or the encoded protein. The expression is preferably downregulated. For example, the change in regulation may be 2-fold, 3-fold, 5-fold, 10-fold, 20-fold, 50-fold, or even 100-fold or more.

The invention also provides a method for identifying a patient as susceptible to treatment with a SLUG, OVOL1 and/or OVOL2-modulating antibody, comprising measuring the expression level of a SLUG, OVOL1 and/or OVOL2 expression product in a biological sample from that patient.

Furthermore, the invention provides a method for identifying a patient as susceptible to treatment with a SLUG, OVOL1 and/or OVOL2-modulating antibody, comprising measuring the expression level of a SLUG, OVOL1 and/or OVOL2 expression product in a biological sample from that patient. In such a method, the expression level of the SLUG, OVOL1 and/or OVOL2 expression product at a first time point may be compared to the expression of the same expression product at a second time point, wherein an increase in expression at the second time point relative to the first time point is indicative of the progression of cancer. The increase or decrease between time points may be 2-fold, 3-fold, 5-fold, 10-fold, 20-fold, 50-fold, or even 100-fold or more.

Standard abbreviations for nucleotides and amino acids are used in this specification.

The practice of the present invention will employ, unless otherwise indicated, conventional techniques of molecular biology, microbiology, recombinant DNA technology and immunology, which are within the skill of those working in the art.

Such techniques are explained fully in the literature. Examples of particularly suitable texts for consultation include the following: Sambrook Molecular Cloning; A Laboratory Manual, Second Edition (2000); DNA Cloning, Volumes I and II (D. N Glover ed. 1985); Oligonucleotide Synthesis (M. J. Gait ed. 1984); Nucleic Acid Hybridization (B. D. Hames & S. J. Higgins eds. 1984); Transcription and Translation (B. D. Hames & S. J. Higgins eds. 1984); Animal Cell Culture (R. I. Freshney ed. 1986); Immobilized Cells and Enzymes (IRL Press, 1986); B. Perbal, A Practical Guide to Molecular Cloning (1984); the Methods in Enzymology series (Academic Press, Inc.), especially volumes 154 & 155; Gene Transfer Vectors for Mammalian Cells (J. H. Miller and M. P. Calos eds. 1987, Cold Spring Harbor Laboratory); Immunochemical Methods in Cell and Molecular Biology (Mayer and Walker, eds. 1987, Academic Press, London); Scopes, (1987) Protein Purification: Principles and Practice, Second Edition (Springer Verlag, N.Y.); and Handbook of Experimental Immunology, Volumes I-IV (D. M. Weir and C. C. Blackwell eds. 1986).

The invention will now be described by way of example only with reference to the following figures. It will be appreciated that modification of detail may be made without departing from the scope of the invention.

### BRIEF DESCRIPTION OF THE DRAWINGS

FIG. 1: BCR-ABL p210 Transcription in a Mouse Model of Chronic Myeloid Leukaemia

FIG. 1 shows the percentage of BCR-ABL p210 transcripts measured in the mouse model of chronic myeloid leukaemia (CML), either in a Sca1⁺Lin⁻ or a Sca1⁻Lin⁺ background, before and after the clinical detectability of CML, as well as after treatment with Gleevec (STI571).

FIG. 2: Survival Study of 70 Metastatic Breast Carcinoma Patients

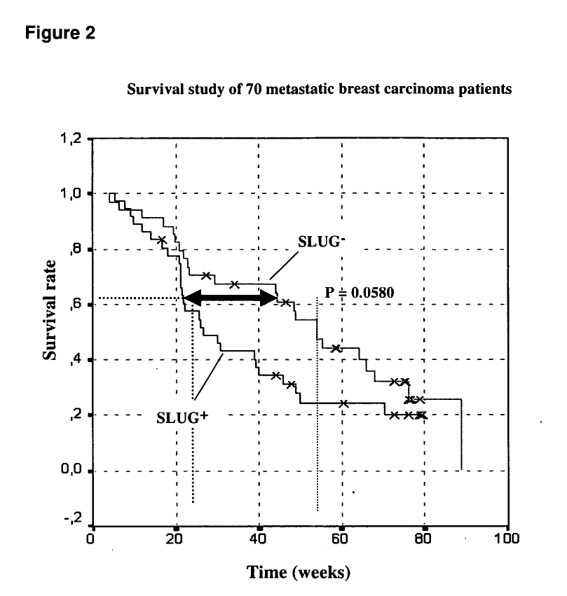

In FIG. 2 it is shown that the survival rate of patients expressing SLUG (SLUG-positive, SLUG⁺ patients) was significantly lower than the survival rate of SLUG-negative (SLUG⁻) patients (P=0.0580).

### EXAMPLES

### Example 1

### Materials and Methods

1.1 Flow Cytometry

Nucleated cells were prepared from peripheral blood cell suspensions. In order to further prepare cells for flow cytometry, contaminating red blood cells were lysed with 8.3% ammonium chloride and the remaining cells were then washed in PBS with 2% foetal calf serum (FCS). After staining, all cells were washed once in PBS with 2% FCS containing 2 μg/mL propidium iodide (PI) to allow dead cells to be excluded from both analyses and sorting procedures. Monoclonal antibodies were obtained from Pharmingen and included: antibodies against CD45R/B220, CD19, Ly51, CD43, IgM and IgD for B lineage staining; antibodies against CD4, CD8 and CD3 for T cell lineage; antibodies against CD11b and Gr1 for myeloid lineage and Sca1 for stem cell staining. Single cell suspensions from the different tissue samples obtained by routine techniques were incubated with purified anti-mouse CD32/CD16 (Pharmingen) to block binding via Fc receptors and with an appropriate dilution of the different antibodies at room temperature or 4° C., respectively. The samples and the data were analysed in a FACScan apparatus using CellQuest software (Becton Dickinson). The specific fluorescence of fluorescein isothiocyanate (FITC) and PE was excited at 488 nm (0.4 W) and 633 nm (30 mW), respectively. Known forward and orthogonal light scattering properties of mouse cells were used with established gates. For each analysis, at least 5.000 viable (PI⁻) cells were assessed.

1.2 Treatment of Animals With STI571 (Gleevec)

For the animal studies, stock solutions of 5 mg/mL and 10 mg/ml were prepared fresh in water, sterile filtered and administered to mice in a volume of 250 μl by gavage twice a day. Mice were started on STI571 or placebo (the same volume of diluent water) beginning on day after leukaemia was confirmed (day 0) by means of an STI571 regimen of 50 mg/kg every morning and 100 mg/kg every evening by gavage. STI571 was administered in a volume of 250 μl sterile water by means of straight or curved animal feeding needles. Mice tolerated the therapy well and no interruption of therapy was necessary. Mice were clinically examined 3 times a week, and periodic peripheral blood counts were obtained by tail vein blood draw as indicated. For the survival analysis portion of this study, the death endpoint was determined either by spontaneous death of the animal or by elective killing of the animal because of signs of pain or suffering according to established criteria.

1.3 Real-Time PCR Quantification

To analyse expression of BCR-ABL p210, reverse transcription (RT) was performed according to the manufacturer's protocol in a 20-μl reaction containing 50 ng of random hexamers, 3 μg of total RNA, and 200 units of Superscript II RNase H-free (RNase H⁻) reverse transcriptase (GIBCO BRL). Real-time quantitative PCR was carried out for the quantitation of BCR-ABL p210. Fluorogenic PCRs were set up in a reaction volume of 50 μl using the TaqMan PCR Core Reagent kit (PE Biosystems). cDNA amplifications were carried out using the same primers in a 96-well reaction plate format in a PE Applied Biosystems 5700 Sequence Detector. Thermal cycling was initiated with a first denaturation step of 10 min at 95° C. The subsequent thermal profile was 40 cycles of 95° C. for 15 s, 56° C. for 30 s, 72° C. for 1 min. Multiple negative water blanks were tested and a calibration curve determined in parallel with each analysis. The abl endogenous control (PE Biosystems) was included to relate BCR-ABL p210 to total cDNA in each sample. The sequences of the specific primers and probe for abl were as follows:

| | |
|---|---|
| sense primer | 5′-CACTCTCAGCATCACTAAAGGTGAA-3′, |
| antisense primer | 5′-CGTTTGGGCTTCACACCATT-3′, |
| and | |
| probe | 5′-CCGGGTCTTGGGTTATAATCACAATG-3′. |

### Example 2

### Identification of Cancer Stem Cells (CSC) as a Biomarker For the Prediction and Monitoring of Cancer in a Sca1/BCR-ABL p210 Mouse Model

Chronic myeloid leukaemia (CML) was used as a model to test whether cancer stem cells (CSC) may be used as a biomarker for various aspects of cancer. CML was modelled in mice. The use of CSC as biomarkers, in particular, in the monitoring and/or prediction of cancer development, dissemination and relapse was then tested in these mice.

Mouse CSC were obtained by introducing human cancer-associated genetic alterations into Sca1+ cells in mice. In the present example, BCR-ABL p210 was expressed in Sca1+ cells. The CSC were thus Sca1+ cells expressing BCR-ABL p210. Expression of BCR-ABL p210 was measured as described in Example 1.3.

The presence of these CSC in the peripheral blood of Sca1/BCR-ABL p210 mice was monitored before the onset of CML, once CML was detected and in mice treated with Gleevec (STI571). Treatment with Gleevec was effected as described in Example 1.2.

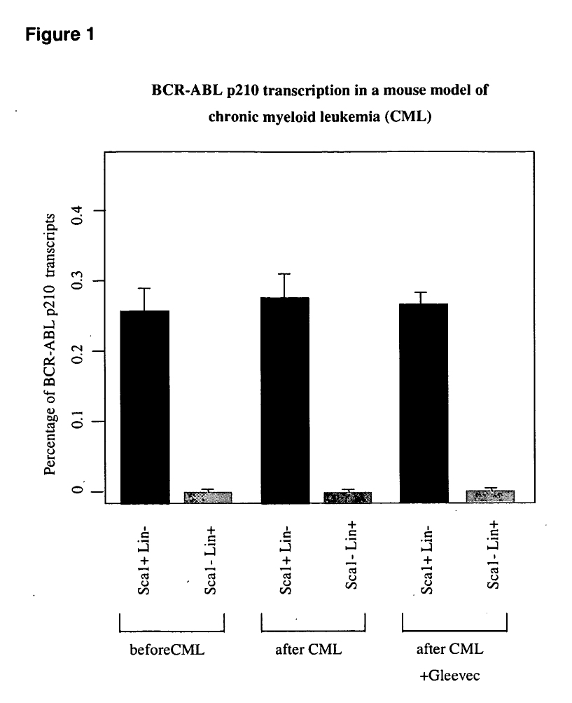

The following results were obtained (see also FIG. 1):

1) Before cancer (chronic myeloid leukaemia, CML) could be clinically detected, BCR-ABL p210 transcripts were observed in a Sca1+Lin− background, but not in a Sca1−Lin+ background, and CSC were present in the peripheral blood of all Sca1/BCR-ABL p210 mice.

2) Once CML could be clinically detected, BCR-ABL p210 was observed in a Sca1+Lin− background and not in a Sca1−Lin+ background, and CSC were present in peripheral blood of Sca1/BCR-ABL p210 mice.

3) After treatment with Gleevec, BCR-ABL p210 was again observed in a Sca1+Lin− background and not in a Sca1−Lin+ background. Sca1/BCR-ABL p210 mice did not respond to Gleevec treatment. The detection of CSC in the peripheral blood of these mice during Gleevec treatment indicated failure of the treatment.

Overall, these data showed that CSC in peripheral blood may be used as biomarkers i) to predict cancer development in mice, ii) to monitor dissemination of cancer and iii) to predict and monitor relapse after treatment.

### Example 3

### Identification of Cancer Stem Cells (CSC) as a Biomarker for the Prediction and Monitoring of Cancer in a Sca1/Bcl6 Mouse Model

The procedure of Example 2 was followed, with the exception that Bcl6 was used in the place of BCR-ABL p210. Equivalent results were obtained with Sca1/Bcl6 mice as with the Sca1/BCR-ABL p210 mice of Example 2.

### Example 4

### Identification of Cancer Stem Cells (CSC) as a Biomarker for the Prediction and Monitoring of Cancer in a Sca1/K-RASv12 Mouse Model

The procedure of Examples 2 and 3 was followed, with the exception that K-RASv12 was used in the place of BCR-ABL p210 or Bcl6, respectively. Equivalent results were obtained with Sca1/K-RASv12 mice as with the Sca1/Bcl6 and Sca1/BCR-ABL p210 mice of Examples 2 and 3, respectively.

### Example 5

### Identification of SLUG (Snai2) as a Marker of CSC Using Sca1/BCR-ABL p21 Mice

The expression of SLUG (Snai2) was analysed in different cell types (Sca1+Lin− and Sca1−Lin+) in the peripheral blood of controls and Sca1 mice. This analysis was carried out by reverse-transcription PCR (RT-PCR).

Reverse transcription was performed according to the manufacturer's protocol in a 20-μl reaction containing 50 ng of random hexamers, 3 μg of total RNA and 200 units of Superscript II RNase H-free (RNase H−) reverse transcriptase (GIBCO BRL). The thermocycling parameters for the PCR reactions were as follows: 30 cycles at 94° C. for 1 min, 56° C. for 1 min and 72° C. for 2 min The PCR primers used for amplification of SLUG were

Forward: 5′-GCCTCCAAAAAGCCAAACTA-3′
Reverse: 5′-CACAGTGATGGGGCTGTATG-3′

Amplifications of β-actin RNA served as a control to assess the quality of each RNA sample. The PCR products were confirmed by hybridisation with specific internal probes.

Results are summarised in Table 1. In Sca1/BCR-ABL p210 mice (Sca+Lin− background), SLUG (Snai2) was expressed before the onset of CML, after CML was detected and after treatment with Gleevec. In control mice, and in a Sca1−Lin+ background, no expression of SLUG (Snai2) was detected.

These results indicated that SLUG (Snai2) is a CSC marker. Since CSCs are a marker for cancer, this leads to the conclusion that SLUG (Snai2) is a marker for cancer.

### Example 6

### Identification of SLUG (Snai2) as a Marker of CSC Using Sca1/Bcl6 Mice

The procedure of Example 5 was followed, with the exception that Bcl6 was used in the place of BCR-ABL p210. Equivalent results were obtained with Sca1/Bcl6 mice as with the Sca1/BCR-ABL p210 mice of Example 2.

### Example 7

### Identification of SLUG (Snai2) as a Marker of CSC Using Sca1/K-RASv12 Mice

The procedure of Examples 5 and 6 was followed, with the exception that K-RASv12 was used in the place of BCR-ABL p210 or Bcl6, respectively. Equivalent results were obtained with Sca1/K-RASv12 mice as with the Sca1/BCR-ABL p210 and Sca1/Bcl6 mice of Examples 5 and 6, respectively.

### Example 8

### Measurement of Circulating CSC By Detection of SLUG (Snai2) Expression in Human Breast Cancer Patients

A peripheral blood sample was taken from 55 breast carcinoma patients before the first chemotherapy cycle. A further sample was taken after completion of the chemotherapy regimen. If the cancer was metastatic, a further sample was taken before a new cycle of chemotherapy was initiated.

SLUG (Snai2) was measured by RT-PCR as described in Example 5.

The results before chemotherapy are summarised in Table 2. Of the 55 patients included in the study, 36 tested SLUG-negative and 19 tested SLUG-positive.

### Example 9

### SLUG Detection in Non-Metastatic Breast Carcinoma Patients Undergoing Chemotherapy

Peripheral blood samples were taken from 172 patients with non-metastasising breast carcinomas undergoing chemotherapy. SLUG (Snai2) was measured by RT-PCR as described in Example 5.

Of the 172 patients tested, 88 (51.2%) expressed SLUG and 84 (48.8%) did not (Table 3).

These patients were further monitored and, of the relapses noted up to the date of filing, 75% occurred in patients who were SLUG-positive at diagnosis. Representative examples are provided in Table 6.

### Example 10

### SLUG Detection in Metastatic Breast Carcinoma Patients

Peripheral blood samples were taken from 70 patients with metastasising breast carcinomas. SLUG (Snai2) was measured by RT-PCR as described in Example 5.

Of the 70 patients tested, 36 (51.4%) expressed SLUG and 34 (48.6%) did not (Table 4).

### Example 11

### Survival Study of Metastatic Breast Carcinoma Patients

Progression-free survival of the 70 metastatic breast carcinoma patients of Example 10 was monitored over a period of 80 weeks, according to the presence of SLUG-positive cells in the peripheral blood. SLUG (Snai2) was measured by RT-PCR as described in Example 5.

As is shown in FIG. 2, survival rate of SLUG-positive (SLUG+) patients was significantly lower than the survival rate of SLUG-negative (SLUG−) patients (P=0.0580 for a survival rate of about 0.6 at around 26 and 53 weeks for SLUG+ and SLUG− patients, respectively ).

Results of a multivariate statistical analysis of the risk of progression for the 70 metastatic patients are provided in Table 5. Overall, it was found that the risk of progression was 3.226 (1/0.310) times higher in SLUG+ patients than in SLUG− patients. The risk of progression was 2.2 times higher in patients with previous treatments than in patients without previous treatments, and 10 times higher in patients with three or more metastases than in patients with no metastases.

### Example 12

### Measurement and Therapeutic Implications of Circulating CSC in Lung Carcinoma Patients

The same protocol was followed as for breast carcinoma patients. 60 patients were analysed, of which all (100%) were smokers. SLUG+ cells were detected in 53.33% of these patients.

### Example 13

### Measurement and Therapeutic Implications of Circulating CSC in Ovarian Carcinoma Patients

The same protocol was followed as for breast and lung carcinoma patients. 10 patients were analysed. SLUG+ cells were detected in 80% of these patients.

### Example 14

### Identification of OVOL1 and OVOL2 as a Marker of CSC Using Sca1/BCR-ABL p21 Mice

The expression of OVOL1 and OVOL2 was analysed in different cell types (Sca1+Lin− and Sca1−Lin+) in the peripheral blood of controls and Sca1 mice. This analysis was carried out by reverse-transcription PCR (RT-PCR).

RT-PCR was performed according to the manufacturer's protocol in a 20-μl reaction containing 50 ng of random hexamers, 3 μg of total RNA, and 200 units of Superscript II RNase H− reverse transcriptase (GIBCO/BRL). The thermocycling parameters for the PCR reactions using specific primers were as follow: 30 cycles at 94° C. for 1 min, 56° C. for 1 min, and 72° C. for 2 min. The primers used for the amplification of OVOL1 and OVOL2 were:

OVOL1
Forward: 5′-AGCTGTGACCTGTGTGCCAA-3′
Reverse: 5′-ACTCAGGCTGGTATTCTCCT-3′
OVOL1
Forward: 5′-TCGAGACTCTAGCTACAGCA-3′
Reverse: 5′-AGTGACAGTGTTCTGTAGTG-3′

Amplification of β-actin RNA served as a control to assess the quality of each RNA sample. The PCR products were confirmed by hybridization with specific internal probes.

Results are summarised in Table 7. In Sca1/BCR-ABL p210 mice (Sca+Lin− background), OVOL1 and OVOL2 were in CML, B-cell lymphoma, Multiple meyloma and lung carcinoma. In control mice, and in a Sca1−Lin+ background, no expression of OVOL1 or OVOL2 was detected.

These results indicated that OVOL1 and OVOL2 are a CSC markers. Since CSC are a marker for cancer, this leads to the conclusion that OVOL1 and OVOL2 are markers for cancer.

### Example 15

### Measurement of Circulating CSC By Detection of OVOL1 and OVOL2 Expression in Human Breast Cancer Patients

Peripheral blood samples were taken from 32 patients breast carcinomas who were positive for SLUG (Snai2) expression as measured by RT-PCR (described in Example 5).

OVOL1 and OVOL2 expression was measured in peripheral blood samples as described in Example 14.

Of the 32 patents, 4 (12.5%) were positive for OVOL1 expression and 13 (40.6%) were positive for OVOL 2 expression, as shown in Table 8.

### Example 16

### Measurement of OVOL1, OVOL2 and hSLUG Expression in Healthy Controls

Peripheral blood samples were taken from healthy control 105 patients.

OVOL1 and OVOL2 expression was measured in peripheral blood samples as described in Example 14. SLUG expression was measured in peripheral blood samples as described in Example 5.

Of the 105 patents, 0 were positive for OVOL1 expression and 3 were both positive for OVOL 2 expression and negative for hSLUG expression, as shown in Table 9.

### Example 17

### Measurement of hSLUG Expression in Patients With Lung Carcinoma

Peripheral blood samples were taken from 62 patients with lung carcinomas. SLUG (Snai2) was measured by RT-PCR as described in Example 5.

Of the 62 patients tested, 32 (51.6%) expressed SLUG and 30 (48.4%) did not (Table 10).

### Example 18

### Measurement of hSLUG Expression in Patients With Metastatic Ovarian Carcinoma

Peripheral blood samples were taken from 42 patients with metastatic ovarian carcinomas. SLUG (Snai2) was measured by RT-PCR as described in Example 5.

Of the 42 patients tested, 33 (78.6%) expressed SLUG and 9 (21.4%) did not (Table 11).

### Example 19

### Measurement of hSLUG Expression in Patients With Colon Carcinoma

Peripheral blood samples were taken from 41 patients with colon carcinomas. SLUG (Snai2) was measured by RT-PCR as described in Example 5.

Of the 41 patients tested, 26 (63.4%) expressed SLUG and 15 (36.6%) did not (Table 12).

TABLE 1
SLUG (Snai2) is a marker of cancer stem cells in mice

| | Sca1/BCR-ABL p210 mice | | | | | |
|---|---|---|---|---|---|---|
| control | before CML | | after CML | | AfterCML + Gleevec | |
| mice | Sca1⁺Lin⁻ | Sca1⁻Lin⁺ | Sca1⁺Lin⁻ | Sca1⁻Lin⁺ | Sca1⁺Lin⁻ | Sca1⁻Lin⁺ |
| −− | ++ | −− | ++ | −− | ++ | −− |

TABLE 2
SLUG expression in 55 breast carcinoma patients before chemotherapy

| SLUG | Patients | Percentage |
|---|---|---|
| negative | 36 | 65.5 |
| positive | 19 | 34.4 |
| total | 55 | 100.0 |

TABLE 3
SLUG expression in 172 non-metastatic breast
carcinoma patients during chemotherapy

| SLUG | Patients | Percentage |
|---|---|---|
| negative | 84 | 48.8 |
| positive | 88 | 51.2 |
| total | 172 | 100.0 |

TABLE 4
SLUG expression in 70 metastatic breast carcinoma patients

| SLUG | Patients | Percentage |
|---|---|---|
| negative | 34 | 48.6 |
| positive | 36 | 51.4 |
| total | 70 | 100.0 |

TABLE 5
Multivariate analysis of the risk of progression
of 70 metastatic breast carcinoma patients

| | | | 95.0% confidence interval | |
|---|---|---|---|---|
| | | | for (1/R.R.) | |
| | | 1/R.R. | Inferior | Superior |
| | p-value | (#) | limit | limit |
| SLUG | 0.016 | 0.310 | 0.119 | 0.805 |
| Previous treatment | 0.005 | 0.453 | 0.182 | 0.856 |
| Number of metastases | 0.50 | 0.102 | 0.010 | 0.999 |
| (≧3) | | | | |

(#) R.R.: relative risk

TABLE 6
Relapses in SLUG+ patients

| Patient | | | | | | Size | Age | | Date of | Date of |
|---|---|---|---|---|---|---|---|---|---|---|
| code | TNM | ER | PR | CerbB2 | Nodes | (cm) | (years) | SLUG | diagnosis | relapse |
| 411 | T1N0M0 | − | − | +++ | 0 | 0.6 | 48 | Positive at | 1/6/2004 | 30/8/2005 |
| | | | | | | | | diagnosis | | |
| 465 | T1N0M0 | − | − | − | 0 | 2 | 66 | Positive at | 25/9/2004 | 23/5/2005 |
| | | | | | | | | diagnosis | | |
| 470 | T1N0M0 | − | + | + | 0 | 2 | 37 | Positive at | 21/9/2004 | 19/8/2005 |
| | | | | | | | | diagnosis | | |
| 518 | T2N0M0 | − | − | − | 0 | 2 | 43 | Positive at | 21/10/2004 | 9/1/2006 |
| | | | | | | | | diagnosis | | |

TNM: (Tumour, nodes, metastases staging scale)
ER: Oestrogen receptor
PR: Progesterone receptor
CerbB2 CerB2/HER2 protein
Nodes: Adenopathies or nodes

TABLE 7
OVOL1 and OVOL2 are CSC markers in mice

| | | B-cell | Multiple | Lung |
|---|---|---|---|---|
| | CML | lymphoma | myeloma | carcinoma |
| Sca1 + Lin− cells | OVOL1+ | OVOL1+ | OVOL1+ | OVOL1+ |
| | OVOL2+ | OVOL2+ | OVOL2+ | OVOL2+ |
| Sca1 − Lin+ cells | OVOL1− | OVOL1− | OVOL1− | OVOL1− |
| | OVOL2− | OVOL2− | OVOL2− | OVOL2− |
| control mice | OVOL1− | OVOL1− | OVOL1− | OVOL1− |
| | OVOL2− | OVOL2− | OVOL2− | OVOL2− |

TABLE 8
OVOL1 and OVOL2 expression in breast carcinoma patients
with hSLUG expression in the peripheral blood.

| | | |
|---|---|---|
| OVOL1 | negative | 28 patients |
| | positive | 4 patients |
| OVOL2 | negative | 19 patients |
| | positive | 13 patients |
| hSLUG | negative | 0 patients |
| | positive | 32 patients |

TABLE 9
OVOL1, OVOL2 and hSLUG expression the
peripheral blood of healthy controls.

| | | |
|---|---|---|
| OVOL1 | negative | 105 patients |
| | positive | 0 patients |
| OVOL2 | negative | 102 patients |
| | positive | 3 patients |
| hSLUG | negative | 3 patients |
| | positive | 102 patients |

TABLE 10
hSLUG expression in the peripheral blood of Lung carcinoma patients

| | | |
|---|---|---|
| hSLUG | negative | 30 patients |
| | positive | 32 patients |

TABLE 11
hSLUG expression in the peripheral blood of ovarian
carcinoma patients with metastatic disease

| | | |
|---|---|---|
| hSLUG | negative | 9 patients |
| | positive | 33 patients |

TABLE 12
hSLUG expression in the peripheral blood of colon carcinoma

| | | |
|---|---|---|
| hSLUG | negative | 15 patients |
| | positive | 26 patients |

1. A method of detecting, identifying and/or quantifying cancer stem cells, the method comprising the step of assessing:
(a) the level of expression;
(b) the activity; or
(c) the sequence
of the OVOL1 gene, promoter and/or expression product in a cell.

2. A method according to claim 1, wherein the cancer stem cells are haematopoietic, epidermal, breast, ovary, lung, pancreas, prostate, brain, colon, bone marrow, and/or lymph cancer stem cells.

3-5. (canceled)

6. A method for the eradication of cancer stem cells, said method including the step of
(a) immunotherapy directed at cancer stem cells by targeting SLUG, OVOL1 and/or OVOL2 expression products or their coding genes or promoters; or
(b) inducing a switch in cancer stem cells either to effect a change from exponential to non-exponential cell growth or to induce a differentiation or apoptotic program.

7-9. (canceled)

10. A method according to claim 1, wherein the expression product of OVOL1 is mRNA.

11. A method according to claim 10, wherein the mRNA is detected using Northern Blot, RT-PCR or qRT-PCR.

12. A method according to claim 1, wherein the expression product of OVOL1 is a protein.

13. A method according to claim 12, wherein the protein is detected by an antibody.

14. A method according to claim 1 that is carried out in vitro.

15-16. (canceled)

17. A purified nucleic acid which comprises the promoter of the SLUG, OVOL1 and/or OVOL2 gene operatively linked to a heterologous gene, wherein said heterologous gene is:
(a) an oncogene; or
(b) a reporter gene.

18. A transgenic non-human animal which comprises a heterologous nucleic acid construct that comprises the OVOL1 promoter and/or coding sequence.

19. A transgenic non-human animal according to claim 18, wherein the nucleic acid construct comprises the nucleic acid of claim 17.

20. The use of a transgenic non-human animal of claim 18 to model human cancer and/or other stem cell derived diseases.

21. A method of detecting cancer stem cells in a transgenic non-human animal, comprising the steps of identifying the cells in the transgenic non-human animal of claim 19 that express the reporter gene.

22. A substantially pure culture of cancer stem cells, wherein said cells express OVOL1.

23-24. (canceled)

25. The purified cancer stem cell culture of claim 22, wherein the cancer stem cells are derived from epithelial cancer and/or mesenchymal cancer.

26. The purified cancer stem cell culture of claim 25, wherein the epithelial cancer and/or mesenchymal cancer is a lymphoma, leukaemia, sarcoma and carcinoma, or wherein the epithelial cancer and/or mesenchymal cancer is chronic myeloid leukaemia, B-cell acute lymphoblastic leukaemia, T-cell acute lymphoblastic leukaemia, acute myeloid leukaemia, chronic myeloid leukaemia, lymphoproliferative syndromes, multiple myeloma, liposarcoma, and Ewing sarcoma.

27. (canceled)

28. A method of isolating cancer stem cells comprising the selective enrichment of cells which express OVOL1.

29. A method involving propagating the cancer stem cell of claim 22.

30. A method for identifying a candidate agents that modulate the growth and/or development of a cancer stem cell, comprising:
(a) detecting the level of expression of an expression product of the OVOL1 gene or promoter in the presence of the candidate agent; and
(b) comparing that level of expression with the level of expression in the absence of the candidate agent, wherein a reduction in expression indicates that the candidate agent modulates the level of expression of the expression product of the OVOL1 gene or promoter.

31-32. (canceled)

33. A method for treating cancer in a patient, comprising reducing the number of cancer stem cells that express a product of the OVOL1 gene, said method comprising the step of administering to the patient an antibody, a nucleic acid, a polypeptide or agent identified by the method of claim 30 in a therapeutically-effective amount sufficient to target cancer stem cells for destruction.

34. (canceled)

35. A method of localising and/or imaging cancer stem cells using a radiolabelled antibody that binds specifically to the OVOL1 protein or a radiolabelled nucleic acid probe that hybridises specifically with the OVOL1 mRNA, coding sequence and/or promoter sequence

36. The method of claim 35, wherein the imaging is carried out using PET, CT, SPECT or NMR.


SEQUENCE LISTING (9 sequences)

SEQ ID NO: 1 - DNA, 25 bp, Artificial Sequence
  synthetic
cactctcagcatcactaaaggtgaa

SEQ ID NO: 2 - DNA, 20 bp, Artificial Sequence
  synthetic
cgtttgggcttcacaccatt

SEQ ID NO: 3 - DNA, 26 bp, Artificial Sequence
  synthetic
ccgggtcttgggttataatcacaatg

SEQ ID NO: 4 - DNA, 20 bp, Artificial Sequence
  synthetic
gcctccaaaaagccaaacta

SEQ ID NO: 5 - DNA, 20 bp, Artificial Sequence
  synthetic
cacagtgatggggctgtatg

SEQ ID NO: 6 - DNA, 20 bp, Artificial Sequence
  synthetic
agctgtgacctgtgtgccaa

SEQ ID NO: 7 - DNA, 20 bp, Artificial Sequence
  synthetic
actcaggctggtattctcct

SEQ ID NO: 8 - DNA, 20 bp, Artificial Sequence
  synthetic
tcgagactctagctacagca

SEQ ID NO: 9 - DNA, 20 bp, Artificial Sequence
  synthetic
agtgacagtgttctgtagtg